(19) United States
(12) Patent Application Publication
(10) Pub. No.: US 2023/0012151 A1
(43) Pub. Date: Jan. 12, 2023

(54) METHOD AND SYSTEM FOR IMPROVING TREATMENT ADHERENCE LEVEL

(71) Applicant: MEDHELPER INC., Pointe-Claire (CA)

(72) Inventors: Terrence Fitzpatrick, Saint-Lazare, QC (CA); Frederic Simard, Saint-Lambert, QC (CA)

(21) Appl. No.: 17/757,119

(22) PCT Filed: Dec. 9, 2020
(86) PCT No.: PCT/CA2020/051696

§ 371 Date: Jun. 9, 2022

Related U.S. Application Data
(60) Provisional application No. 62/945,845, filed on Dec. 9, 2019.

(51) Int. Cl. G16H 20/10; G16H 40/20; G16H 15/00

(52) U.S. Cl. CPC G16H 20/10; G16H 40/20; G16H 15/00

(57) ABSTRACT

A computer-implemented method to improve adherence level to a medical treatment plan or medication regimen is disclosed. The method comprises the steps to integrate with the payer, to create or manage a care plan, to engage caregivers, to connect to pharmacies and to engage with HCPs. The method may also comprise connection with one or more communities or may comprise connection with external resources. A system for improving treatment adherence level is disclosed. The system comprises a server, computerized devices for patients, computerized devices for caregivers and a network configured to allow communication between the different devices and stakeholders. The computerized devices for patients are configured to fetch and display treatment or medications plan data associated with the user. The computerized devices for caregivers are configured to fetch and display treatment or medication plans data associated with the one or more users (patients) being followed by the caregiver.

### CROSS-REFERENCE TO RELATED APPLICATIONS

The present patent application claims the benefits of priority of United States Pro-visional Patent Application No. 62/945,845, entitled “METHOD AND SYSTEM FOR IMPROVING TREATMENT ADHERENCE LEVEL” and filed at the United States Patent and Trademark Office on Dec. 9, 2019, the content of which is in-corporated herein by reference.

### FIELD OF THE INVENTION

The present invention generally relates to methods and systems aiming at improving medical treatment adherence level. More particularly, the present invention relates to methods and systems to improve medical treatment adherence level using computer-implemented methods and computer systems.

### BACKGROUND OF THE INVENTION

Systems for helping patients or individuals following a medical treatment plan or a medication regimen have been known for many years. Such systems typically provide the user with automatic reminders to improve adherence to the aforementioned treatment or medication. Separate systems provide access to certain elements of the patient data, such as displaying and/or paying drug scripts, creating checklist of activities to complete as part of the treatment plans, or even scheduling appointments with health care professionals. Each of those systems generally help patients with a single aspect of their treatment or medication plan. Also, such systems are non-connected native applications which do not allow the exchange of data with other external or 3rd party systems.

There is therefore a need for a system configured to improve adherence of patients to their medical treatment plan or medications that covers all aspects of adherence to said plans and by interconnecting such aspects of adherence.

### SUMMARY OF INVENTION

The shortcomings of the prior art are generally mitigated by providing a method and a system for improving treatment adherence level of patients.

In one aspect of the invention, a computer-implemented method to improve adherence level to a medical treatment or medication plan is provided. The method comprises the steps to integrate with the payer, to create or manage a care plan, to engage caregivers, to connect to pharmacies and to engage with HCPs.

The method may further comprise connection with one or more communities or may comprise connection with external resources.

In another aspect of the invention, a system for improving treatment adherence level is provided. The system comprises a server configured to execute steps of the method as described above, computerized devices for patients, computerized devices for caregivers, a network configured to allow communication between the different devices. The computerized devices for patients are configured to fetch and display treatment or medication plan data associated with the user. The computerized devices for the patient are further configured to monitor actions of the user with regard to the said treatment or medications plans, such as the time and frequency of medications intake by the patient, time and frequency of any other activities by the patient, etc.

The computerized devices for caregivers are further configured to request access to the data of the patient user and to fetch and display treatment or medication plan data associated with the one or more users being followed by the caregiver.

Other and further aspects and advantages of the present invention will be obvious upon an understanding of the illustrative embodiments about to be described or will be indicated in the appended claims, and various advantages not referred to herein will occur to one skilled in the art upon employment of the invention in practice.

### BRIEF DESCRIPTION OF THE DRAWINGS

The above and other aspects, features and advantages of the invention will become more readily apparent from the following description, reference being made to the ac-companying drawings in which:

FIG. 1 is an illustration of exemplary elements used in a method and system for improving treatment adherence level in accordance with the principles of the present invention.

FIG. 2 is an architecture diagram of an embodiment of a system for improving treatment adherence level in accordance with the principles of the present invention.

FIG. 3 is an exemplary screenshot of prescription of a care plan using the system of FIG. 2.

FIG. 4 is an exemplary screenshot of an application improving adherence to a prescribed care plan using the system of FIG. 2.

FIG. 5 is an exemplary screenshot of a method to accept to retrieve information from a user using the system of FIG. 2.

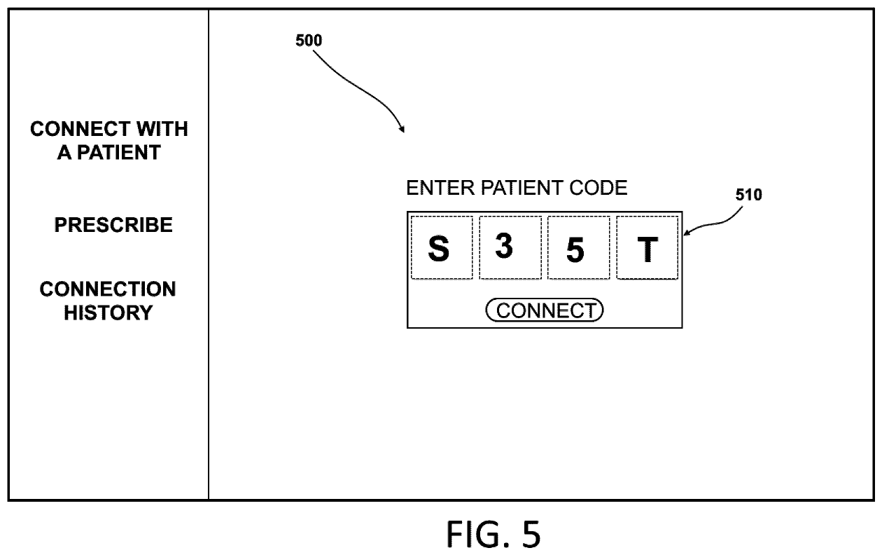

FIG. 6 is an exemplary report of the adherence result and treatment details generated by a method and system for improving treatment adherence level in accordance with the principles of the present invention.

### DETAILED DESCRIPTION OF THE PREFERRED EMBODIMENT

A novel method and system for improving treatment adherence level will be described hereinafter. Although the invention is described in terms of specific illustrative embodiments, it is to be understood that the embodiments described herein are by way of example only and that the scope of the invention is not intended to be limited thereby.

The present invention generally aims at driving greater medication and treatment adherence than any other approach available today. As such, a unique methodology based on “closing the loop” between key elements impacting the patients is disclosed. The methodology allows these elements to be brought together in a single environment to enable patient achieving better compliance behavior to their medications or treatment plans;

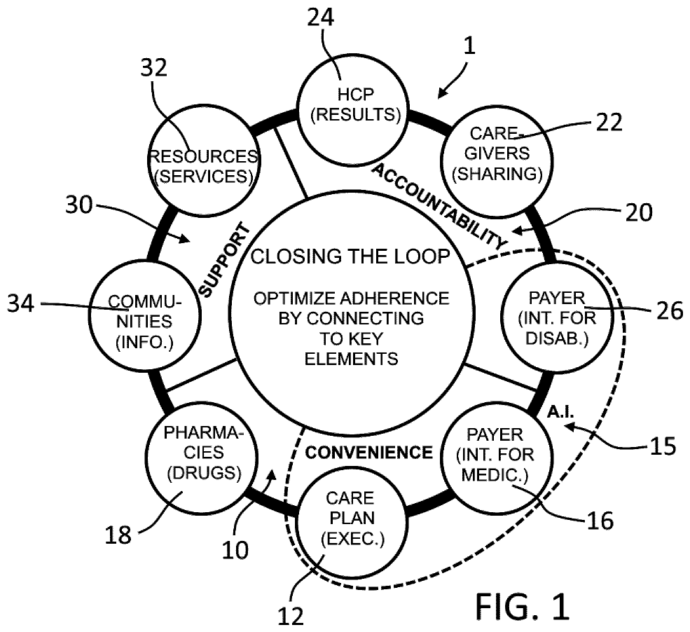

Referring now to FIG. 1, an illustration of exemplary elements used in an embodiment of the method and system for improving treatment adherence level is illustrated. The present method 1 broadly aims at increasing the medication and treatment adherence level of patients or any person following a medical treatment by bringing together different elements in a single environment. Such method 1 further aims at helping users achieving better compliance behaviors with medications or treatment plans.

One type of elements is the payer 16, 26 of health services provided to the patient. Non limiting examples of payers 16, 26 may be workers compensation boards, government agencies, insurance companies, etc. The payers generally provide funds for ill patients, or patients on short- or long-term disability treatments.

Another type of element is a care plan 12 which broadly represents the medications or treatment components that a patient is asked to follow, comply with or adhere to in order to follow or comply with instructions from one or more health care professionals.

Caregivers 22 represent third parties, such as but not limited to family members, loved ones, professional service workers and/or nurses, being involved in a supervision role of the user care and compliance to the treatment or medication plan. The caregivers 22 are given access to data relating to actions taken by the user to comply with the medication or medical treatment plan. In some embodiments, the caregiver 22 executes a computer program on a device having access to the user's data.

A further element are the pharmacies or drug suppliers 18. The pharmacies 18 shall procure medication using various efficient means.

Health care professionals 24 (HCP) comprise an engagement with the patient or user for sharing information, such as but not limited to doctor report, physiology feedback, etc. and system integration, such as EMR, drug and treatment import, etc.

Other elements may comprise communities 34, meaning engagement of external communities 34. As an example, the method 1 may comprises integration with a community of local diabetes patients using a mobile application or website.

Another element may be the resources 32, meaning the procurement of services in support of the treatment or medication plan of the user or patient, such as but not limited to wellness program, virtual care, etc.

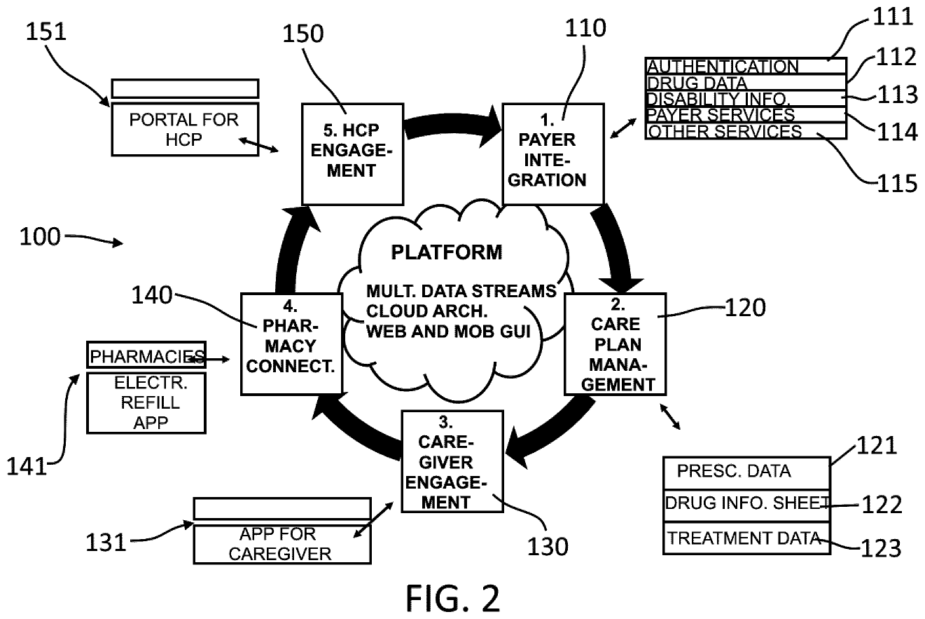

Referring now to FIG. 2, a computer-implemented method 100 to improve adherence level to a medical treatment or medication plan is illustrated. The method comprises the steps to integrate with the payer 110, to create a care plan 120, to engage the caregivers 130, to connect to pharmacies 140 and to engage with HCPs 150.

The method 100 may further comprise connection with one or more communities 160 or may comprise connection with external resources 170.

The step to integrate with the payer 110 may comprise authenticating with an authentication system 111 of one or more payers 16 or 26. In some embodiments, a computer program comprising instructions to be executed by a computer, such as a server, is configured to connect with the authentication system of one or more payers 16 or 26 through a network. The computer program may be configured to provide a token or authentication data of the user or patient to the payer 16 or 26 system.

The integration with the payer 110 may further comprise fetching the medication or medical treatment data 112 from the payer 16 or 26. In some embodiments, a server or computer is configured to connect or communicate with a server or system of the payer 102 to retrieve patient's data, such as medication/treatment information. In some implementations, the server of the payer comprises application programming interfaces (API) or any other mean of data exchange allowing access to the patient's data. As an example, the server may connect to an insurance claims repository 16 of the payer or a disability management system 26 of the payer.

The integration with the payer 110 may further comprise fetching the disability data of patient 113 from the payer 26. Again, in some embodiments, a server or computer is configured to connect or communicate with a server or system of the payer to retrieve the patient's disability data. In some implementations, the server of the payer comprises application programming interfaces (API) or any other mean of data exchange allowing access to the patient's disability data.

The integration with the payer 110 may further comprise providing other services of the payer 114 through a single interface, such in an application used by the patient user or other partner services 115.

The integration with one or more payer 110 generally aims at optimizing the patient adoption and engagement by combining such integration 110 with the care plan management 120.

The step to create a care plan or to manage a care plan 120 may further comprise importing the fetched medication information 121, such as prescription data, or the treatment information 123 from the payer in the server. Understandably, the server may be hosted either in a local computer environment or in any known cloud-type computer environment.

The creation of a care plan 120 may further comprise fetching and storing additional medication information into the server 121. In some embodiments, the system is configured to retrieve prescription data of the patient from a pharmacy or any other remote system comprising such prescription data. In yet other embodiments, the system is configured to retrieve detailed drug information sheet 122, such as using 3rd party providers allowing access to detailed medication data.

The creation of a care plan 120 may further comprise capturing and/or processing treatment information from HCPs 123. In some embodiments, HCPs may comprise doctors, nurses, physical therapists, or any other type of healthcare professionals. In some exemplary implementations, a portal program 151 may be configured to connect with any systems of the HCPs or to manually enter or transfer the information from HCPs data sources. The server may be configured to communicate the patient data with the portal program.

The creation of a care plan 120 may also comprise combining data from the various source of data, such as importing the fetched medication information or the treatment information from the payer, prescription data, detailed drug information and/or treatment information from the HCPs. Such combination generally aims at creating a centralized user experience, such as providing such information in a single interface of an application, such as a mobile application or a web application.

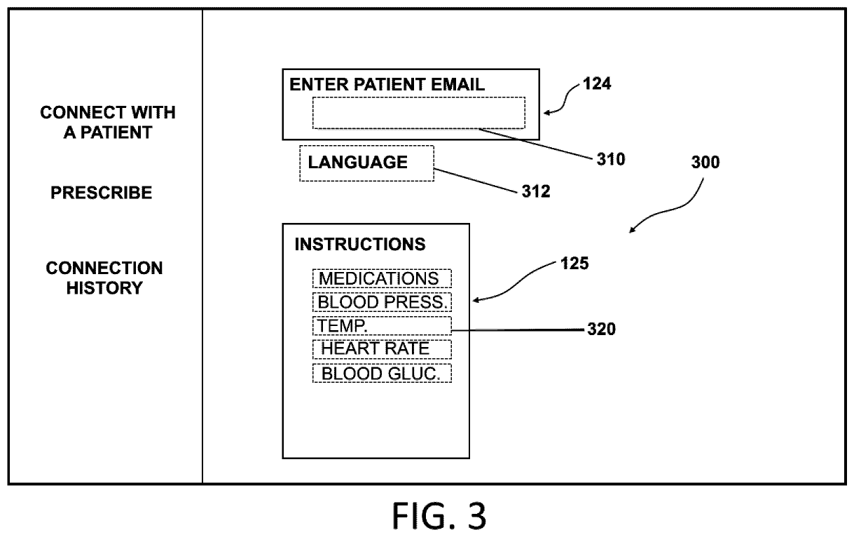

Referring now to FIG. 3, an exemplary graphical user interface (GUI) 300 to create a care plan 120 is illustrated. In such embodiment, the step to create a care plan 120 may comprise identifying the patient 124 and selecting the instructions or parameters of the care plan 125.

The identification of the patient 124 generally comprises inputting a unique identifier 310, such as but not limited to an email address, phone number, etc. The identification of the patient 124 may further comprise specifying parameters related to the patient 312, such as identifying language of communication.

The step of selecting the instructions may further comprise selecting the specific instructions 320 to add to the care plan 12, such as but not limited to following medications, following custom or specific activities, following blood pressure and/or heart rate, temperature, respiratory rate, oxygen saturation, blood glucose and/or identifying specific pain.

In yet other embodiments, the step of selecting the instructions 320 may further comprise uploading or linking documents associated to the care plan, adding manually entered instructions from the HCP, selecting doses, frequencies, or other parameters of the medications or of prescribed activities.

Typically, the HCP identifies the patient 124, select the instructions 125 and save the care plan 12. In some embodiments, upon saving the care plan, a request is sent to a server to save and/or store the created care plan. The server is generally configured to notify the patient of the new care plan. In some embodiments, the patient or user receive a notification, typically through an email address.

In some embodiments, the server is configured to identify if the user is registered or not. If the patient is not registered with the system, a link to a registration page or to download an application is provided in the said email or communication. In other embodiments, the server may be further configured to create an incomplete account associated with the unique identifier 310. The incomplete account is populated with the created care plan 12. The account may be labeled as incomplete or as a ghost account. Upon acceptation of the invitation to the care plan 12, the server activates the account for the specific patient.

If the patient is already registered, the server may be configured to send a notification in the account of the patient or an application in communication with a server. The server may further be configured to create or update a care plan 12 in the account of the registered patient.

The patient accepts the created care plan 12 through the sent link or communication. The accepted care plan 12 is then accessible to the patient, typically through an application installed on a computerized device, through a web portal or any known system for presenting a care plan to a user. The management of care plan 120 general aims at assisting the patient to improve patient adoption and engagement. As such the management of care plan 120 may comprise using schedule or reminders in relation to medication plan or treatment plan. Understandably, any known method or system configured to provide schedules or reminders may be used within the scope of the present invention.

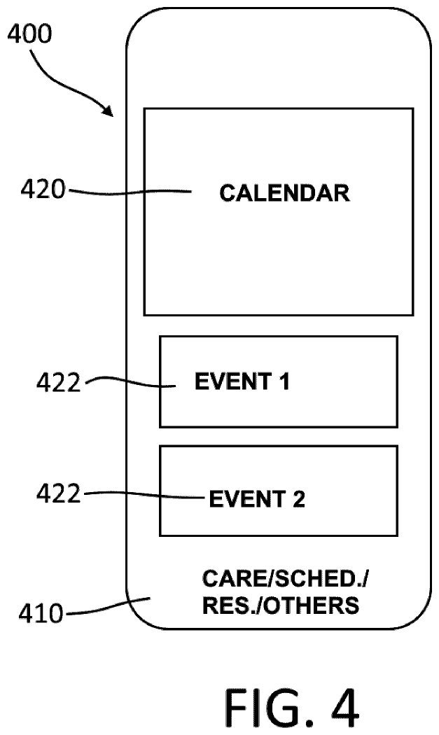

Referring now to FIG. 4, an exemplary GUI of an application to execute a care plan 12 is shown. In such embodiment, an application is executed on a computerized device 410 of the care giver or patient. In such embodiments, a calendar 420 and the different care plan 12 instructions for a specific time period 422 are displayed. One skilled in the art should understand that the computerized device may be configured to enable any features needed or desirable for the execution of a care plan 12 which are known in the art.

The step to engage caregivers 130 may further comprise processing the patient data to identify the behavior of the patient with regard to medication or treatment plan. The step 130 may further comprise displaying a real time view of the identified behavior of the patient adherence to the one or more plans. Such display generally aims at providing a real time view or visibility on the behavior of the patient to another user of the system, such as a user associated with a caregiver. In some embodiment, the computerized device of a caregiver is configured to execute an application or to access a remote application to access patient's data and to act on the engagement of the patient 131, also referred as the companion application.

The step to engage caregivers 130 generally aims at driving desired behavior and providing an oversight of the resulting behavior of the patient.

The step to connect to pharmacies 140 may further comprise displaying inventory of medications and electronically requesting a refill of medications such as to avoid gap in drug possession. In some implementations, the server may be configured to communicate with one or more systems of a pharmacy or a pharmacy group through an API or similar data exchange platform or system.

The step to connect to pharmacies 140 may further comprise sending a request of a temporary dosage change to a pharmacy system. In some embodiments, the server may be configured to send a request of temporary dosage change to the system or data exchange platform of a pharmacy. Such request typically comprises parameters such as but not limited to new dosage details, unique medication ID (e.g. DIN in Canada), inventory on-hand, start date of dosage and end date of dosage.

The step to connect to pharmacies 140 may also comprise transferring one or more scripts of a patient user from local pharmacy to another pharmacy, such as an out of town pharmacy. In some embodiments, the server may be configured to send a request to a local pharmacy system for transferring the script. The request generally comprises the pharmacy where to transfer the script and the out-of-town emergency doses. The server is typically in contact with both pharmacy systems through API or other data exchange systems. The transfer of such information is typically done via electronic means such as but not limited to eRx switching network or within an internal pharmacy software. Understandably, there are many ways of transferring such information such as using any known electronic prescribing system or any electronic system to come.

The step to connect to pharmacies 140 may also comprise requesting one or more electronic refill applications 141. The request generally comprises the pharmacy where to request the refill and the identification of the script associated with the refill. The server is typically in contact with both pharmacy systems through API or other data exchange systems. Again, the request for refill is typically done via electronic means such as but not limited to eRx switching network or within an internal pharmacy software. Understandably, there are many ways of requesting a refill of medication such as using any known electronic prescribing system or any electronic system to come.

The step to connect to pharmacies 140 generally aims at staying current with the medication using online refills.

Referring now to FIG. 51, an exemplary embodiment of GUI for a patient to allow access to data 500 associated or generated through the execution of the care plan 12 is illustrated. In such embodiment, the GUI 500 may require input of a unique generated code 510. In use, the patient requests a unique generated code 510 from the server through but not limited to an application or a web portal. The server generates the unique code 510 and communicate the said code to the computerized device 410 of the patient. When in communication with the HCP, the patient provides the generated code 510 to the HCP. The HCP inputs the code 510 through the GUI 500. The code 510 is communicated to the server. Understandably, any type of code may used such as but not limited to a numeric or alphanumeric code, a bar code, a QR code, b

The server is configured to receive and/or validate the code and, upon validation, to communicate the shared data or information relating to the care plan 12 of the patient to the computerized device of the HCP. The computerized device of the HCP may be configured to input the code 510 through a web portal, an application, or any other known system for communicating with the server.

In some embodiments, the unique code 510 may have a predetermined lifespan. In such embodiments, the server is configured to invalidate the unique code 510 after a predetermined period of time or duration.

The step to engage with HCPs 150 may further comprise processing the data of the patient user into adherence results. The step 150 may further comprise generating a report of the adherence result and treatment details. Such report may be based on a predefined doctor or medical report. In a typical embodiment, the patient may print or present such report to any HCP aiming at expediting the analysis of patient status by the HCP.

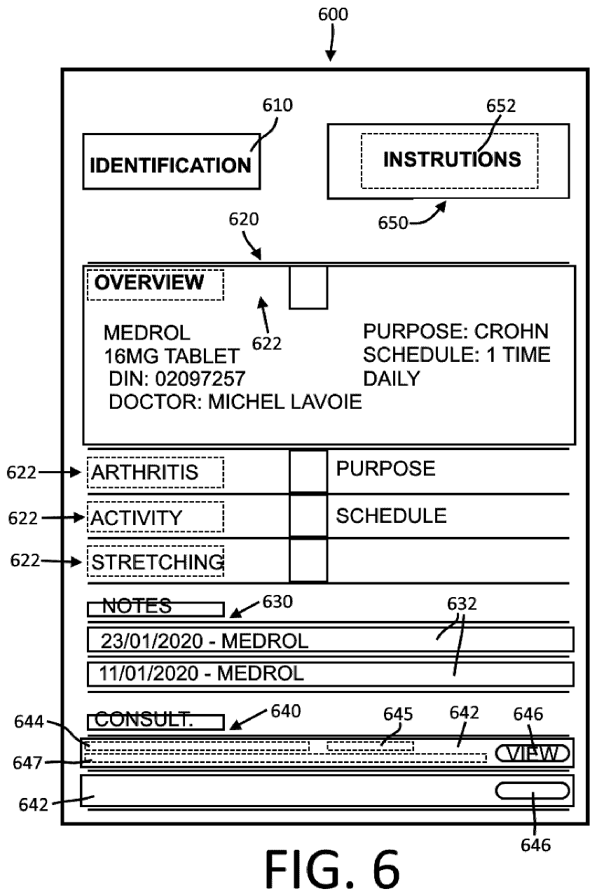

Referring now to FIG. 6, an exemplary report 600 of shared data relating to a care plan 12 is illustrated. The report 600 generally comprises identification 610 of the patient or care plan user, such as but not limited to the name of the patient, a photo, the gender, the age and/or known allergies. Understandably, any other desirable information relating to the patient may be displayed.

The report 600 may further comprise a treatment overview 620. The treatment overview generally comprises the different instructions 622 of the care plan 12. The instructions 622 may relate to activities, medications, etc.

The report 600 may further comprise notes or observations of the patient 630. The details of the notes or observations 632 are generally displayed in a chronological order.

The report 600 may further comprise historical data about the care plan 640, such as the previous consultations details 642. The previous consultations 622 may comprise the consulted HCP or resources 644, the detail of the clinic where the event occurred 645, access to session, notes, or observations of the event 646 and/or manual entries or observations by the HCP 647.

The report 600 may further comprise inputting further instructions 650 to be added to the care plan 12, such as new instructions based on the progress of the care plan 12 or specific data of the report 600. Upon input of the instructions 652, the instructions 652 are communicated to the server. The server is configured to store the instructions 652 and associated the said instructions 652 with the care plan 12 of the patient.

In typical embodiments, the HCP may not change the report 600 or has a read-only access to the report 600.

In yet other embodiments, the HCP may be redirected to the GUI for creating/updating a care plan 300. In such embodiments, the system may be configured to require the patient to provide the HCP with a unique generate code 510 to allow the HCP to change the care plan 12.

The idea to drive adherence by engaging with HCPs 150 is generally threefold. First the HCP report is easily accessible to the patient user and allows to be easily shown or shared with an HCP, thus driving more adherence from the patient user. Second, the data recorded and stored by the system may be used by HCPs to validate treatment effectiveness and recommend changes to the said treatment. Third, the connection with HCPs systems drive adherence by generally improving convenience of the treatment experience, such as but not limited to avoiding trip to doctors by communicating via the platform on side effects and else in cases on chemotherapy.

The step to engage with HCPs 150 may also comprise exchanging patient information or data with a portal accessible only by HCPs 151. In some implementations, the HCP portal may be hosted on a computer configured to either gain access to the patient data through the server or to communicate such data to the server through any communication protocol or any API or similar data exchange platform or system.

The step 150 may further comprise sending a request to the HCP portal to update patient medication or treatment plan for continuous improvement of health outcome. Such request may be prepared and sent by an application accessible to HCP, such as a web application or a mobile application. The request typically comprises the changes about the medication or treatment plan. The server is typically in contact with the HCP system through API or other data exchange systems. In yet other embodiments, the server may communicate with HCP system using any known electronic means such as web-services between various end points including but not limited to an application running on a computerized device for the patient, a portal for HCPs or an administrative portal, such as a web-based application platforms for the HCPs.

The step 150 generally aims at reviewing results and adjusting treatment for continuous improvement, thus restarting a loop using the method and system 100 from the step of integrating with payer 110.

Broadly, the complete loop provides continuous improvement on the adherence level and may be summarized as explained hereinafter. The payers generally provide a tool usable by patients, the tool helping the said user with the management of their treatment plan, easy onboarding with system integration, drug imports, personal data, etc. Such tool of the payer generally aims at driving up the adoption of an adherence tool by patients via integration with the payer 110.

Then, the patient users are responsible for using the tool to manage their care plan, to setup reminders, to record activities, to monitor adherence results. Such usage generally aims at optimizing adherence of the patient user to their treatment via the care plan management 120.

Then, the caregiver users create a dynamic of oversight or supervision via a tool that naturally drives the patient to a more engaged experience. Such oversight generally aims at driving greater treatment compliance by patients via open caregiver engagement step 130.

Next, the patients have access to medications or scripts at all time and may procure themselves with required medications in an effective way via a tool of the system. Such ease of access generally aims at maximizing adherence with greater convenience via the pharmacy connection 140.

Then, by influencing patient user in the face of potential doctor reporting, the desired behavior is generally accelerated or improved. Furthermore, sharing data with HCPs system generally improve treatment quality. Such reporting and sharing of data generally aim at driving adherence and enhancing quality of care via HCP engagement 150.

The loop is then closed or completed as the payer is now in a position with the benefit of better data to enhance the tool's functionalities and offer targeted services which, in turn, will allow the payer to offer better support to the patient on its journey to living a healthier life by being more adherent to its treatment plan and getting better quality care, hence, aiming at closing the loop for continuous improvement.

The step to connect with one or more communities 160 may comprise connecting or linking patient users to one or more group of users of the system having or sharing common interests. In some implementations, the server may be configured to analyze the data associated to each user and to segment matching elements of data from all or a select group of users, such as geographical information, diseases, medications, etc. In some embodiments, the server is configured to establish a connection to a community platform, such as ZenDesk®.

The step to connect with external resources 170 may further comprise storing a list of partners providing additional or complementary healthcare services, such as but not limited to wellness programs, virtual care, etc. The step further comprises displaying the list of partners in a graphical user interface. In some embodiments, the server is configured to connect and communicate with 3rd party partners, such as through an electronic communication channel or by connecting to API provided by the 3rd party partner.

In some embodiments, a system for improving treatment adherence level is provided. The system comprises a server configured to execute steps of the method 100 as described above. The server typically comprises a central processing unit (CPU), transient memory, a storage unit, and a network adapter.

The system further comprises computerized devices for patients adapted to execute instructions to connect and communicate with the server through a network. The computerized devices for patients generally comprise a CPU, a display unit, transient memory, a network adapter, and a storage unit. The computerized devices for patients are further configured to fetch and display treatment or medications plans data associated with the user. The computerized devices for the patient are further configured to monitor actions of the user with regard to the said treatment or medications plans, such as the time and frequency of medications intake by the patient, time and frequency of any other activities by the patient, etc.

In some embodiments, the system is implemented as a distributed system, where each component may be hosted in different location or may be positioned in different locations. As an example, the system of the payer may be hosted at a first location, the server of the present system in a second location, the computerized device for the patient user may be located in a third location and the computerized device for the caregiver may be located in a fourth location. Understandably, all components may be communicating through one or more data networks.

The system further comprises computerized devices for caregivers. The computerized devices for caregivers generally comprise a CPU, a display unit, transient memory, a network adapter, and a storage unit. The computerized devices for caregivers are configured to request access to the patient user data, to visualize patient data, and exchange information with the patient device. In a typical embodiment, the computerized device for caregiver sends a request to the server comprising the patient user identification and the caregiver identification. The server then sends a request to the patient user, such as to inform the patient device. The patient computerized device is configured to receive the request, to display the caregiver information requesting the sharing and to provide a mechanism to accept the caregiver request and notify the acceptation via the server. Upon acceptation of the request, the server establishes a connection between the patient and the caregiver devices.

The computerized devices for caregivers are further configured to fetch and display treatment or medications plans data associated with the one or more users being followed by the caregiver.

In some other embodiments, when more than one caregiver is linked to a patient user, the computerized device of caregivers may be configured to create tasks and to assign tasks to other caregivers, aiming at distributing and spreading the caregiving tasks between all the caregivers. In such embodiments, the computerized device of a patient may be further configured to assign a main caregiver in charge of creating the tasks and to assign the tasks to other caregivers.

In some other embodiments, the computerized device of caregivers may be configured to capture expenses relating to caregiving activities in the form of a ledger or, in some embodiments, related to a task as described above. In such embodiments, the server may be configured to store the expense and to generate an expense ledger or report for a caregiver user based on predetermined parameters, such as start date, end dates, type of expenses, etc.

While illustrative and presently preferred embodiments of the invention have been described in detail hereinabove, it is to be understood that the inventive concepts may be otherwise variously embodied and employed and that the appended claims are intended to be construed to include such variations except insofar as limited by the prior art.

1. A computer-implemented method for improving treatment adherence level of a patient, the method comprising:
integrating with one or more payer system through a network;
creating and updating a care plan for the patient hosted on a server;
increasing level of engagement of one or more caregiver of the patient;
increasing level of engagement of one or more health care professional (HCP) treating the patient.

2. The computer-implemented method of claim 1, the method further comprising connecting with one or more communities of the patient.

3. The computer-implemented method of claim 1, the integration with one or more payer system further comprising automatically authenticating with an authentication system of the one or more payer system through a network.

4. The computer-implemented method of claim 1, the integration with one or more payer system further comprising automatically retrieving the medication or medical treatment data of the patient from the payer system through a network.

5. The computer-implemented method of claim 4, the creation or update of the care plan of the patient further comprising automatically storing the retrieved medication or medical treatment data to data source.

6. The computer-implemented method of claim 1, the integration with one or more payer system further comprising automatically retrieving data regarding disability of the patient from the payer system through a network, the creation or update of the care plan of the patient further comprising automatically storing the retrieved disability data to a data source.

7. The computer-implemented method of claim 1, the method further comprising automatically connecting with a system of a pharmacy to retrieve prescription data of the patient, the creation or update of the care plan of the patient further comprising automatically storing the retrieved prescription data to a data source.

8. The computer-implemented method of claim 7, connecting with the system of the pharmacy further comprising displaying inventory of medications and electronically sending a request to the system of the pharmacy to refill a medication of the retrieved prescription data.

9. The computer-implemented method of claim 7, connecting with the system of the pharmacy further comprising sending a request of a temporary dosage change to the system of the pharmacy.

10. The computer-implemented method of claim 7, connecting with the system of the first pharmacy further comprising sending a request to a system of a second pharmacy system for transferring the script from the first pharmacy.

11. The computer-implemented method of claim 1, the creation or update of the care plan of the patient further comprising the HCP inputting treatment information regarding the patient, the inputted treatment being stored with the care plan of the patient.

12. The computer-implemented method of claim 8, the treatment information being automatically inputted by connecting to an HCP system through a network.

13. The computer-implemented method of claim 8, the treatment information being manually inputted by an HCP through a computer program.

14. The computer-implemented method of claim 1, the creation or update of the care plan of the patient further comprising identifying the patient and inputting instructions or parameters of the care plan.

15. The computer-implemented method of claim 11, the creation or update of the care plan of the patient further comprising identifying the patient using a unique identifier.

16. The computer-implemented method of claim 12, the instructions being selected in followings: documents, medications, custom or specific activities, blood pressure and/or heart rate, temperature, respiratory rate, oxygen saturation, blood glucose or specific pain identification.

17. The computer-implemented method of claim 12, the method further comprising:
storing the created or updated care plan;
notifying a computerized device of the patient of the new or updated care plan using the unique identifier;
the computerized device of the patient accepting the created or updated care plan;
the new or updated care plan being activated for the patient.

18. The computer-implemented method of claim 14, the method further comprising:
creating a temporary account for the patient if the patient is not registered;
associating the created care plan to the account; and
activating the care plan upon registration of the patient.

19. The computer-implemented method of claim 1, the increasing of engagement of the one or more caregiver of the patient further comprising processing the data retrieved regarding the patient to identify behaviors of the patient regarding medication or treatment plan.

20. The computer-implemented method of claim 16, the method further comprising displaying a real time view of the identified behavior of the patient adherence to the one or more care plans.

21. The computer-implemented method of claim 1, the method further comprising allowing access to the care plan to the HCP.

22. The computer-implemented method of claim 21, allowing the access to a care plan to the HCP further comprising:
generating a unique code to the patient;
the HCP entering the unique code using a computer program;
the computer program validating the unique code;
upon validation of the unique code, displaying the care plan to the HCP.

23. The computer-implemented method of claim 22, only shared data of the care plan being displayed to the HCP.

24. The computer-implemented method of claim 22, the generated unique code expiring after a predetermined duration.

25. The computer-implemented method of claim 1, increasing level of engagement of one or more HCP further comprising processing data of the patient into adherence results.

26. The computer-implemented method of claim 25, processing data of the patient into adherence results further comprising generating a report of the adherence results and of the care plan details.

27. The computer-implemented method of claim 1, increasing level of engagement of one or more HCP further comprising inputting further instructions to be added to the care plan of the patient and communicating the added instructions to a server.

28. A system for improving treatment adherence level of patient users, the system comprising:
a server connected to a network, the server comprising a central processing unit, transient memory and a storage unit, the server being configured to:
fetch the data associated with the patient user from one or more payer systems;
receive instructions from a health care professional (HCP) to create or update a care plan associated with one of the patient user;
share at least partially the care plan associated with the patient to the HCP;
computerized devices for the patient users configured to:
fetch from the server and display treatment or medications associated with the care plan associated with the patient user;
monitor actions of the patient regarding the care plan;
communicate with the server through the network;
computerized devices for caregivers configured to:
request access to the patient user data;
visualize the accessible patient data; and
exchange information with the patient computerized device.

29. The system of claim 28, the computerized devices of caregivers being further configured to:
send a sharing request to the server comprising the patient user identification and the caregiver identification;
notify the computerized device of the patient user of the request;
the computerized device of the patient user accepting the request to share data of the patient user to the computerized device of the caregiver;
the server being further configured to establish a connection between the computerized device of the patient and computerized device of the caregiver.

30. The system of claim 29, the computerized devices of caregivers being further configured to fetch and display treatment or medications plans data associated with the one or more patient users being connected to the caregiver.

31. The system of claim 29, the computerized devices of caregivers being further configured to create tasks and to assign tasks to other caregivers to distribute the tasks between a plurality of caregivers.

32. The system of claim 29, the computerized devices of caregivers being further configured to capture expenses relating to caregiving activities in the form of a ledger.

33. The system of claim 32, the server being configured to store the captured expenses and to generate an expense ledger for a caregiver user based on predetermined parameters.

34. The system of claim 28, the system further comprising computerized devices of HCP being further configured to access the care plan of one or more patient users.

35. The system of claim 34, the server being further configured to generate a unique code to access data of the care plan of a patient user and to communicate the unique code to the computerized device of the associated patient user, the computerized devices of HCP being further configured to input the generate unique code provided by the patient user and to communicate the inputted generated unique code to the server.

36. The system of claim 35, the server being further configured to validate the unique generated code and to allow access by the computerized device of the HCP to the care plan data of the patient user.
